(19) United States
(12) Patent Application Publication
(10) Pub. No.: US 2023/0089310 A1
(43) Pub. Date: Mar. 23, 2023

(54) METHOD AND APPARATUS FOR MEASURING THE PROSTATIC URETHRAL LENGTH

(71) Applicant: Prodeon Medical Corporation, Taipei (TW)

(72) Inventors: Chun-Chia Juan, Taipei (TW); Yu-Chen Lin, Taipei (TW); Yu-Shih Weng, Taipei (TW); Yue-Teh Jang, Taipei (TW)

(21) Appl. No.: 17/948,473

(22) Filed: Sep. 20, 2022

Related U.S. Application Data
(60) Provisional application No. 63/246,062, filed on Sep. 20, 2021.

(51) Int. Cl. A61B 5/107; A61B 5/20; A61B 5/00

(52) U.S. Cl. CPC A61B 5/1076; A61B 5/20; A61B 5/6853; A61M 25/10182

(57) ABSTRACT

Devices and methods are disclosed for determining prostatic urethral length. A balloon catheter subassembly is in fluid communication between an inner cavity of a syringe body and an expandable balloon is positioned at a distal end of the balloon catheter subassembly. An adapter secured to the syringe body having a syringe plunger includes a lock that engages the syringe plunger at a predefined position with respect to the syringe body corresponding to a desired inflation state of the expandable balloon. Prostatic urethral length can then be determined using markings indicating distance from the expandable balloon.

### RELATED APPLICATIONS

This application claims priority to U.S. Provisional Application No. 63/246,062, filed Sep. 20, 2021. The priority of this application is expressly claimed, and the disclosure is hereby incorporated by reference in its entirety.

### FIELD OF THE PRESENT DISCLOSURE

This disclosure relates to devices for managing or treating body tissues obstructing a hollow body lumen, such as the prostatic lobe tissues obstructing the urethra.

### BACKGROUND

The prostate is a walnut-shaped gland that wraps around the urethra through which urine is expelled from the bladder and plays a crucial role in the reproductive system of men. Although the gland starts out small, it tends to enlarge as a man ages. An excessively enlarged prostate results in a disease known as benign prostatic hyperplasia (BPH). Benign prostatic hyperplasia (BPH) refers to the abnormal, but non-malignant (non-cancerous) growth of the prostate observed very commonly in aging men. BPH is a chronic condition and is associated with the development of urinary outflow obstruction or luminal narrowing in the prostatic urethra. Bladder outlet obstruction (BOO) refers to a blockage at the base of the bladder that reduces or stops the flow of urine into the urethra and may be secondary to BPH. A range of related disorders referred to collectively as Lower Urinary Tract Symptoms (LUTS) can result, including sexual dysfunction, frequent urination, difficulty in voiding urine, urinary retention, urinary leakage, and urinary tract and bladder infections that worsen as the abnormal growth in the prostate enlarges and progresses.

Surgical procedures provide BPH relief by removing a significant portion the prostate tissue. Several traditional surgical procedures are available, all of which require hospitalization and some form of spinal, epidural, or general anesthesia. Transurethral resection of the prostate (TURP) is the main surgical treatment for BPH and remains the gold standard against which other treatments are compared. Traditional surgical techniques differ in the location of the incision made by the surgeon to access the prostate and in the method by which prostatic tissue is removed. For example, some surgeries use laser energy, heat, or radio frequency to remove tissue from the prostate. They include laser enucleation, photoselective vaporization (PVP), transurethral needle ablation (TUNA) using radiofrequency energy, transurethral microwave thermotherapy (TUMT) and transurethral incision of prostate (TUIP). However, these traditional surgical approaches to the treatment of BPH are invasive, non-reversible, and have significant drawbacks including the placement of a temporary catheter for a few months, risk of infection, loss of sexual function, urinary incontinence, and restenosis—wherein recurring hyperplasia of cells in the prostate regrow to cause a recurrence of the narrowing of the urethra opening and also a recurrence of the LUTS symptoms described above. It is estimated that approximately one in five adults report moderate-to-severe LUTS. These urinary storage and voiding problems substantially decrease the quality of life and is associated with various health conditions.

Although removing prostatic tissue relieves some BPH symptoms, tissue removal by traditional surgical approaches is irreversible and any adverse effects of the surgery may afflict the patient for life or affect the patients' quality of life. Moreover, surgical approaches are associated with the inherent risks from the surgery itself, risk of recurrence from the regrowth of removed prostatic tissue, and, depending on the extent of the disease and the particular surgical approach necessary for an individual patient, can require recovery periods as long as 3 to 6 weeks.

Because of the recognized drawbacks of traditional surgery, less invasive therapies have been developed and, depending on the extent of disease, may be chosen by patients and their physicians as an alternative to lifelong medication or surgery. These less invasive therapies may be suited for those patients not willing or medically not fit to have a surgical procedure performed under general anesthesia. In addition, younger patients also prefer a less invasive, reversible treatment without compromising sexual function, and leave the option of receiving a permanent, non-reversible treatment affecting sexual function at a later age. Further, since less invasive therapies permit treatment in the office or clinic using a local anesthetic, benefits include patient's comfort and healthcare system economy as compared to treatments under general anesthesia in a hospital setting.

Less invasive techniques include transurethral methods that actually remove enlarged prostatic tissue that are generally less traumatic than traditional surgery, but each destroys prostatic tissue and is irreversible. To avoid destroying the prostatic tissue, other therapeutic procedures have been developed that are designed to enlarge the diameter of the prostatic urethra without actual removal of tissue from the prostate gland, such as by implanting a device within the prostatic urethra that is designed to enlarge the diameter of the urethra. A prostatic implant involves a procedure wherein the urologist inserts a small device within the prostatic urethra which is narrowed by enlarged prostatic tissue. Once in place, the implant is designed to expand and help keep the urethra open by pushing out the tissue lobes, while preventing enlarged prostate tissue from total impingement and opening of the urethra. Ideally, prostatic implants eliminate the need to surgically remove prostatic tissue and are expected to reduce the risks of infection, sexual dysfunction, and incontinence, inherent and traditional to even less-invasive, surgical approaches. The procedure may also be designed to be reversible since the implants may be removed and additional surgical treatments may be performed in the future.

Presently, there are a variety of medical devices, such as implants or stents, to aid in the control of urinary outflow obstruction. In order to achieve therapeutic effect with less pain in patient and optimize patient's quality of life, such implants or stents must be accurately sized according to the length of the patient's prostatic urethra. In addition to the suitable size of medical device chosen for intended patients, the length of patient's prostatic urethra is also served as predictive factor for surgical treatment of BPH. In some diagnosis procedure, the length of prostatic urethra is also a key item for check to anticipate the association with the degree of symptoms. Due to differences in individual anatomy, each patient has a different prostate size, so that the length of the urethra inside the prostate is also different. For these and other reasons, measuring the correct prostatic urethra length (PUL) would be desirable, such as before placing the implant in order to select the correct size implant.

Accordingly, it would be desirable to provide a medical device and method for measuring the length of the patient's urethra with features of ease of use, satisfactory accuracy of measurement, being comfortable to patient. This disclosure satisfies these and other needs.

### SUMMARY

This disclosure is directed to a measuring device for determining prostatic urethral length. The measuring device may have a syringe body, a syringe plunger, an adapter secured to the syringe body having a lock that engages the syringe plunger at a predefined position with respect to the syringe body and a balloon catheter subassembly that provides fluid communication between an inner cavity of the syringe body with an expandable balloon positioned at a distal end of the balloon catheter subassembly, wherein the balloon catheter subassembly further comprises markings indicating distance from the expandable balloon.

In one aspect, the predefined position of the syringe plunger corresponds to a desired inflation state of the expandable balloon.

In one aspect, the lock may engage automatically when the syringe plunger is at the predefined position. The lock may be an actuator that is biased by a spring into engagement with at least one notch in the syringe plunger. The actuator may slide radially with respect to the syringe body and have at least one flange that engages the at least one notch in the syringe plunger. In one embodiment, the actuator may have two flanges that are configured to engage opposing notches in the syringe plunger.

In one aspect, the actuator may be a pivoting lever having a projection configured to engage the at least one notch in the syringe plunger.

In one aspect, manual movement of the actuator may be configured to cause disengagement from the at least one notch and allow deflation of the expandable balloon.

In one aspect, the lock may be an actuator such that rotation of the actuator selectively engages the syringe plunger at the predefined position. The syringe plunger may have a cap with at least one extension hook such that the actuator has at least one notch that is rotatable into alignment with the at least one extension hook.

In one aspect, the adapter may be a separate element that is attachable to the syringe body.

This disclosure also includes an adapter for a syringe having a syringe body and a syringe plunger. The adapter may have a lock attachable to the syringe body that engages the syringe plunger at a predefined position of the syringe plunger corresponding to a desired delivery volume.

In one aspect, the lock may engage automatically when the syringe plunger is at the predefined position. The lock may be an actuator that is biased by a spring into engagement with at least one notch formed in the syringe plunger. the actuator may slide radially with respect to the syringe body and have at least one flange that engages the at least one notch formed in the syringe plunger. Alternatively, the actuator may be a pivoting lever having a projection configured to engage the at least one notch formed in the syringe plunger.

In one aspect, the lock may be an actuator such that rotation of the actuator selectively engages the syringe plunger at the predefined position, wherein the syringe plunger has a cap with at least one extension hook and wherein the actuator has at least one notch that is rotatable into alignment with the at least one extension hook.

This disclosure is also directed to a method for determining prostatic urethral length of a patient. The method may involve providing a balloon catheter subassembly having an expandable balloon in fluid communication with an inner cavity of a syringe body with an expandable balloon positioned at a distal end of the balloon catheter subassembly, wherein the balloon catheter subassembly further comprises markings indicating distance from the expandable balloon, advancing the expandable balloon through the patient's prostatic urethra into the patient's bladder, depressing a syringe plunger to inflate the expandable balloon within the patient's bladder, locking the syringe plunger at a predefined position with respect to the syringe body with an adapter, wherein the predefined position of the syringe plunger corresponds to a desired inflation state of the expandable balloon and determining prostatic urethral length based at least in part on comparing the markings indicating distance from the expandable balloon to a target location of the patient.

In one aspect, the syringe plunger may be locked at the predefined position automatically.

In one aspect, the adapter may be attached to the syringe body.

In one aspect, the method may also involve disengaging the lock, deflating the expandable balloon and withdrawing the balloon catheter assembly from the patient.

### BRIEF DESCRIPTION OF THE DRAWINGS

Further features and advantages will become apparent from the following and more particular description of the preferred embodiments of the disclosure, as illustrated in the accompanying drawings, and in which like referenced characters generally refer to the same parts or elements throughout the views, and in which:

FIG. 1 is a cross-section of the male anatomy comprising the lower portion of the bladder, showing the prostatic urethra length as extending from the bladder neck to the verumontanum.

FIG. 2 a schematic view of a prostatic urethra length measuring system according to an embodiment.

FIG. 3 a schematic view of a prostatic urethra length adapter and syringe according to an embodiment.

FIGS. 4-5 are detail schematic views showing engagement of a lock with a syringe plunger according to an embodiment.

FIG. 6 is a schematic view of a distal end of a balloon catheter subassembly according to an embodiment.

FIG. 7 is schematic view showing use of the prostatic urethra length measuring device being used with a cystoscope according to an embodiment.

FIG. 8 is a schematic view indicating disengagement of a lock with a syringe plunger according to an embodiment.

FIGS. 9-13 are schematic views of an adapter for locking a syringe plunger according to an embodiment.

FIGS. 14-19 are schematic views of an adapter for locking a syringe plunger using a pivoting lever according to an embodiment.

FIGS. 20-23 are schematic views of an adapter for locking a syringe plunger using a rotating ring according to an embodiment.

### DETAILED DESCRIPTION

At the outset, it is to be understood that this disclosure is not limited to particularly exemplified materials, architectures, routines, methods or structures as such may vary. Thus, although a number of such options, similar or equivalent to those described herein, can be used in the practice or embodiments of this disclosure, the preferred materials and methods are described herein.

It is also to be understood that the terminology used herein is for the purpose of describing particular embodiments of this disclosure only and is not intended to be limiting.

The detailed description set forth below in connection with the appended drawings is intended as a description of exemplary embodiments of the present disclosure and is not intended to represent the only exemplary embodiments in which the present disclosure can be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the specification. It will be apparent to those skilled in the art that the exemplary embodiments of the specification may be practiced without these specific details. In some instances, well known structures and devices are shown in block diagram form in order to avoid obscuring the novelty of the exemplary embodiments presented herein.

For purposes of convenience and clarity only, directional terms, such as top, bottom, left, right, up, down, over, above, below, beneath, rear, back, and front, may be used with respect to the accompanying drawings. These and similar directional terms should not be construed to limit the scope of the disclosure in any manner.

Unless defined otherwise, all technical and scientific terms used herein have the same meaning as commonly understood by one having ordinary skill in the art to which the disclosure pertains. Moreover, as used in this specification and the appended claims, the singular forms “a,” “an” and “the” include plural referents unless the content clearly dictates otherwise.

Definitions: The terms “therapeutically effective displacement” or “therapeutically effective retraction” or “therapeutically effective expansion”, are used interchangeably herein and refer to an amount of displacement of prostatic tissue proximate to a restricted area of a urethra sufficient to increase the urethral lumen and treat, ameliorate, or prevent the symptoms of benign prostatic hyperplasia (BPH) or comorbid diseases or conditions, including lower urinary tract symptoms (LUTS), bladder outlet obstruction (BOO), benign prostatic obstruction (BPO), wherein the displacement of prostatic tissues exhibits a detectable therapeutic, prophylactic, or inhibitory effect. The effect can be detected by, for example, an improvement in clinical condition, or reduction in symptoms or absence of co-morbidities. Examples of clinical measures include a decrease in the international prostate symptom score (IPSS), reduction in post-void residual (PVR) volume of urine in the bladder after relief or increase in the maximum urinary flow rate (Qmax) or improvement in quality of life (QoL), improvement in sexual health (sexual health inventory for men or SHIM score, men's sexual health questionnaire or MSHQ score) after treatment. The precise distance or volume of the displacement of prostatic tissue will depend upon the subject's body weight, size, and health; the nature and extent of the enlarged or diseased prostatic condition and the size of the implant selected for placement in the patient.

As used herein, a patient “in need of treatment for BPH” is a patient who would benefit from a reduction in the presence of or resulting symptoms of enlarged prostatic tissue caused by a non-malignant enlarging of the prostate gland and related disorders, including LUTS, urinary outflow obstruction symptoms and luminal narrowing of the prostatic urethra. As used herein, the terms “implant” or “expander” or “device” refer to the prosthetic device that is implanted within the prostatic urethra to relieve LUTS associated or caused by BPH.

With respect to orientation of the various structures and anatomical references described herein, the term “proximal” and “distal” are relative to the perspective of the medical professional, such as a urologist, who is manipulating the delivery system of the disclosure to deploy the implants described herein. Accordingly, those features of the delivery system held by the hand of the urologist are at the “proximal” end and the assembled system and the implant, initially in its compressed configuration, is located at the “distal” end of the delivery system.

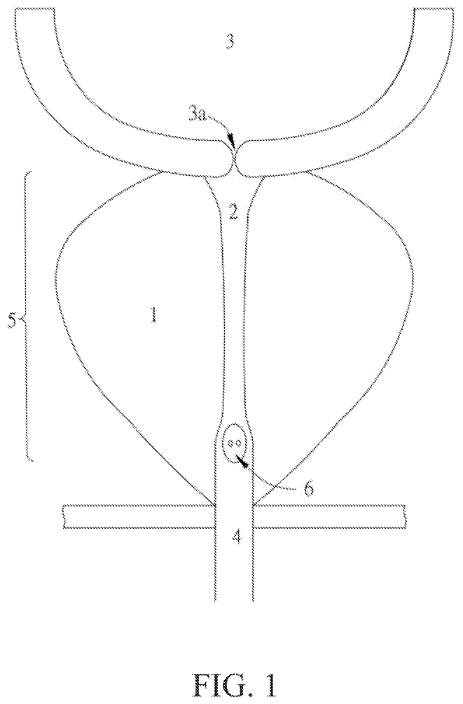

Referring to FIG. 1, a cross-section of the male anatomy shows the prostate gland 1 surrounding the prostatic urethra 2. The prostatic urethra 2, under normal conditions, provides fluid communication from urine stored in the bladder 3 to be expelled from the body under involuntary muscular control of the internal sphincter (or bladder neck) 3a and voluntary muscular control of the external sphincter 4. Normal or “true” prostate tissue of gland 1 surrounds the prostatic urethra 2 and, in the absence of disease, does not impinge on the patency of prostatic urethra 2. As noted above, it is desirable to determine prostatic urethral length (PUL) 5, which for the purposes of this disclosure may be defined as the distance between bladder neck 3a and verumontanum 6.

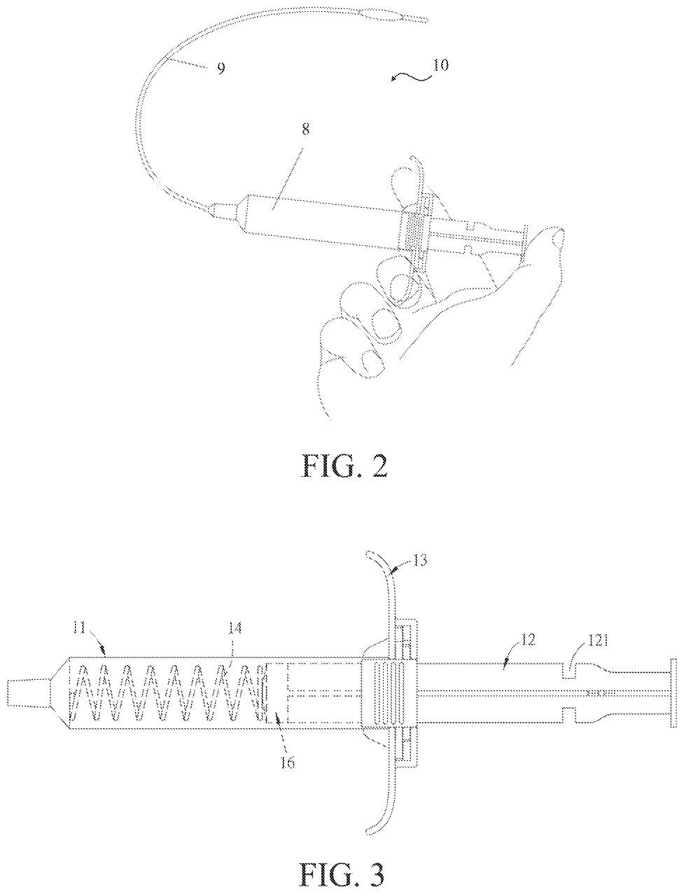

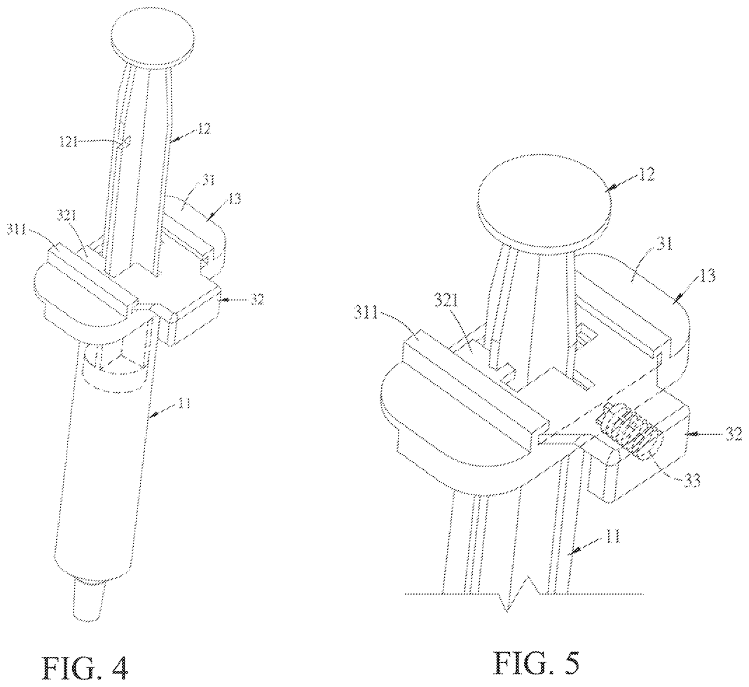

According to an embodiment of this disclosure, a medical device and method of measuring PUL 5 is disclosed. For example, in reference to FIGS. 2 and 3, a suitable system may include a syringe 8 that is in fluid communication with a balloon catheter subassembly 9, forming PUL measurement device 10. As will be discussed in further detail below, PUL measurement device 10 is configured so that syringe 8 includes an adapter to facilitate the control of inflation and deflation of an expandable balloon carried by balloon catheter subassembly 9 as part of a system for measuring PUL from a target location in a subject. In some embodiments, the adapter is configured to be retrofitted to a conventional syringe.

In particular, FIG. 3 is side view of handle 8 of PUL measurement device 10 and depicts a syringe body 11, a syringe plunger 12, a PUL handle adapter 13 and an optional plunger retracting spring 14. As shown, syringe body 11 has a cylindrical cavity and may be made of transparent material. Syringe plunger 12 is generally rod-shaped and is configured to drive a seal 16 located at the distal end of syringe plunger 12. Seal 16 closely conforms to the inner cavity wall of syringe body 11 to reduce and substantially prevent leakage of gas or liquid so that the inner cavity volume of syringe body 11 can be changed by advancing or retracting syringe plunger 12. Syringe plunger 12 may further include at least one notch 121 to implement a locking feature when syringe plunger 12 is at a predetermined position with respect to syringe body 11 representing delivery of a desired amount of inflation fluid or gas. PUL handle adapter 13 interacts with syringe plunger 12 to control the locking and releasing function in cooperation with notch 121. Syringe plunger 12 can be maintained in a specific position by engagement of PUL handle adapter 13 and with notch(es) 121. Syringe body 11 and the expandable balloon form a closed system so that changing the inner cavity volume governs inflation and deflation of the expandable balloon. As such, one or more desired volumes can be selected depending on placement of one or more notches 121 on the outer wall of syringe plunger 12 according to the inner diameter of syringe body 11, stroke of syringe plunger 12, and size of the expandable balloon on balloon catheter subassembly 9. Syringe body 11 may have one or more markers to provide a volume indication. If desired, plunger retracting spring 14 within syringe body 11 may provide an additional retraction force. In some embodiments, syringe body 11 and syringe plunger 12 may be general purpose devices and the one or more notches 121 may be created by an appropriate process such as die cutting or computer number controlled (CNC) machining to relate specific position(s) of syringe plunger 12 to one or more desired volumes. Similarly, adapter 13 may be configured as a separate assembly that can be attached to a conventional syringe body 11 and plunger 12 to provide the locking and releasing functionality. Accordingly, the techniques of this disclosure provide injection and evacuation of either gas or liquid into the distal balloon catheter subassembly with single hand operation. These techniques can also be extended to usage with other devices such as Foley catheter or in other fields of application. If desired, a one-way valve may be located between syringe body 11 and balloon catheter subassembly 9 to help retain an inflated profile of the balloon.

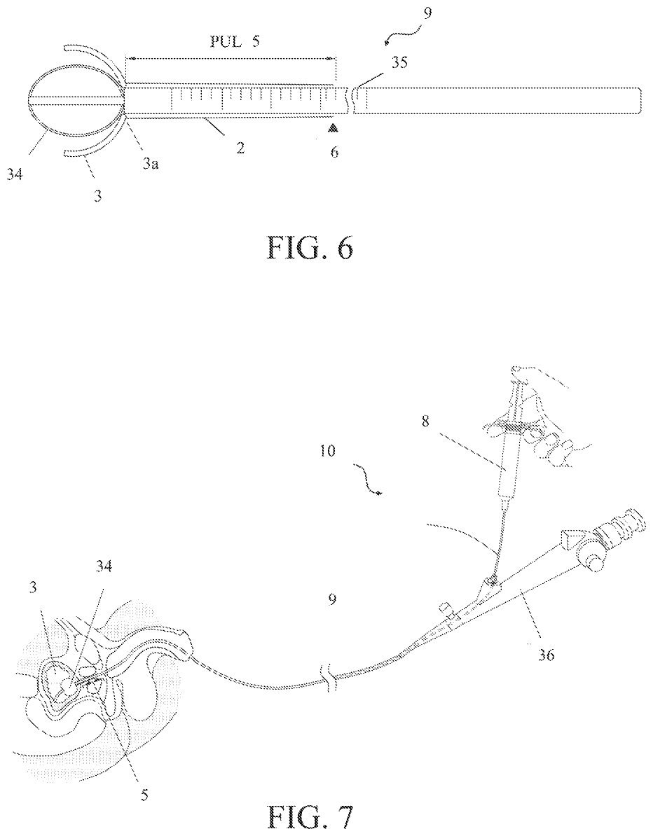

Further details regarding one embodiment of PUL handle adapter 13 are depicted in FIGS. 4 and 5. In particular, PUL handle adapter 13 is shown as including a syringe adapter 31, an actuator 32 and a locking spring 33. Actuator 32 slides along grooves formed in syringe adapter 31 by rails 311 and locking spring 33 biases actuator 32 radially away from syringe plunger 12. A flange 321 prevents the biased movement of actuator 32 unless aligned with the one or more notches 121. For example, as shown in FIG. 4 flange 321 is not aligned with notch 121 and syringe plunger 12 may be advanced or retracted with syringe body 11 but when syringe plunger 12 has been depressed sufficiently as shown in FIG. 5, locking spring 33 biases actuator 32 outwards so that flange 321 engages notch 121 to prevent movement syringe plunger 12 within syringe body 11. As noted above, the volume of the inner cavity of syringe body 11 when flange 321 engages notch 121 corresponds to a desired inflation state of the expandable balloon. Further, spring 33 causes flange 321 to automatically engage notch 121 when syringe plunger 12 is advanced to the predetermined position.

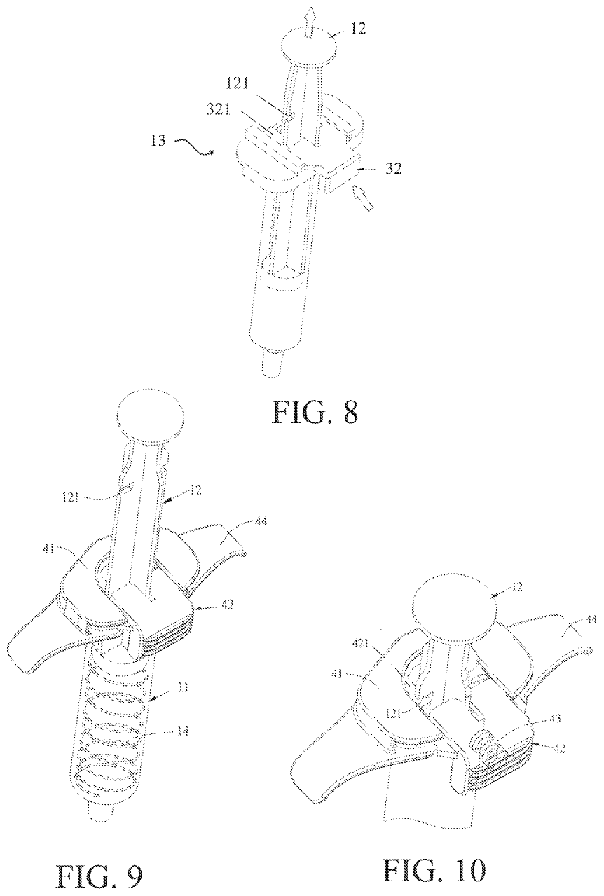

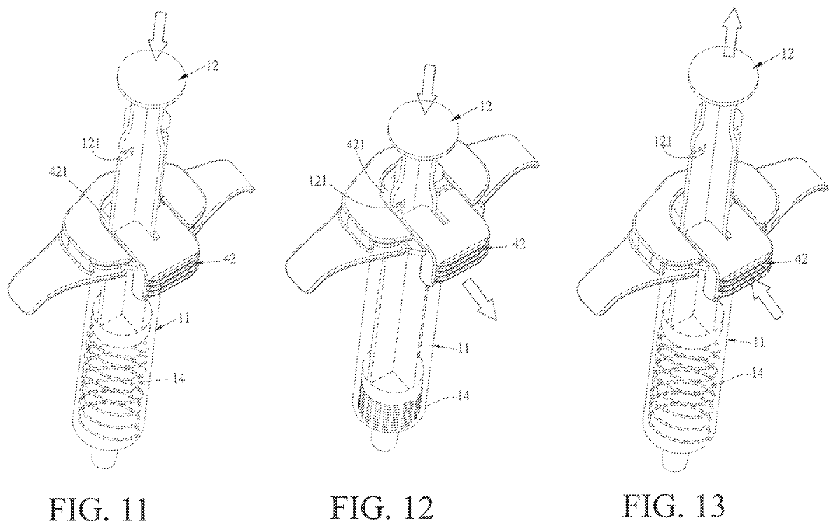

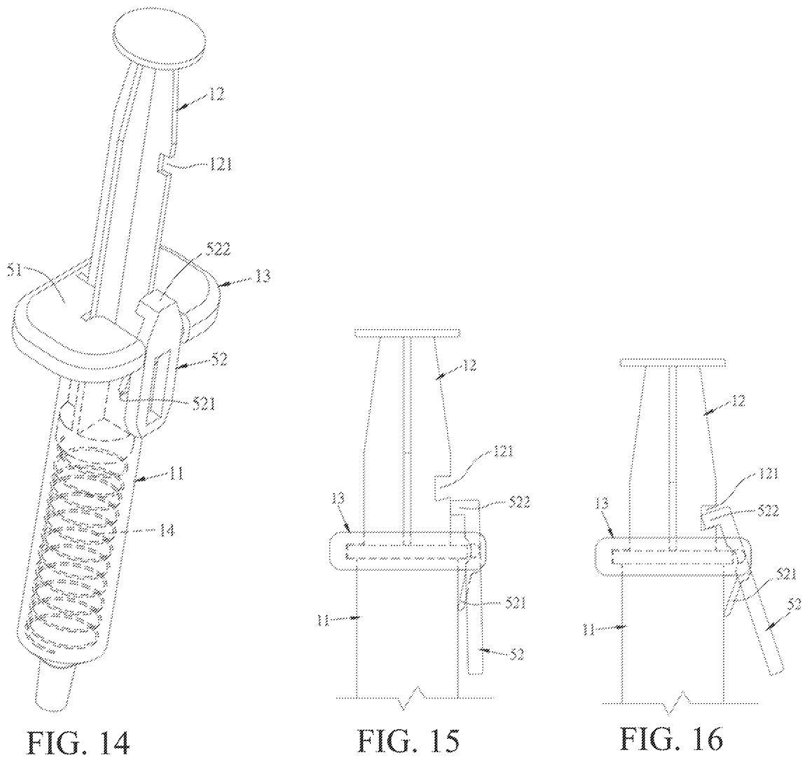

As shown in FIG. 6, balloon catheter subassembly 9 is configured to be advanced through a patient's prostatic urethra 2 so that expandable balloon 34 is positioned within bladder 3. Graduated markings 35 are provided on catheter 36 so that PUL 5 may be readily measured when expandable balloon 34 is inflated and positioned against bladder 3 with a corresponding reading taken for the position of verumontanum 6, such as under visualization via a cystoscope. For example, FIG. 7 schematically depicts an exemplary method of using PUL measuring device 10. As shown, balloon catheter subassembly 9 is advanced through a working channel of cystoscope 36 until balloon 34 is located with bladder 3. Syringe plunger 12 is depressed until notch 121 is engages with flange 321 of actuator 32 as discussed above to maintain balloon 34 at a desired inflation state. Correspondingly, PUL measuring device 10 can be withdrawn until balloon 34 is against bladder 3 at bladder neck 3a and PUL 5 can be measured using the position of verumontanum 6 with reference to markings 35. Following PUL measurement, depressing actuator 32 as schematically depicted in FIG. 8 disengages flange 321 from notch 121 so that plunger 12 can be retracted, optionally with the additional assistance of retracting spring 14 and/or elasticity of balloon 34, to deflate balloon 34 so that it can be withdrawn through the patient's prostatic urethra 2.

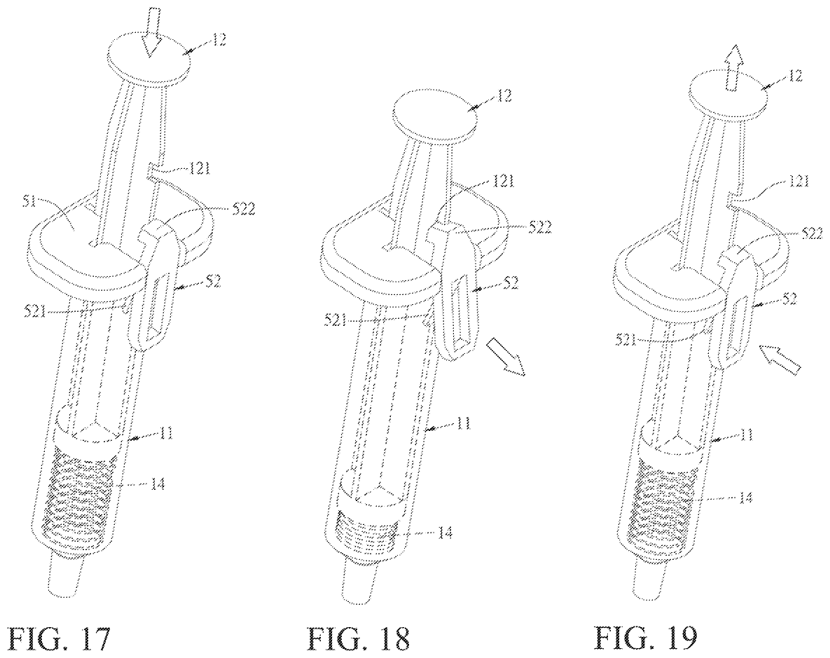

Another embodiment of PUL handle adapter 13 is depicted in FIG. 9. As shown, syringe plunger 12 has opposing notches 121 that are engaged by syringe adapter 41 via actuator 42. Syringe adapter 41 also features extensions 44 in the form of wings or similar structures to facilitate manipulation by the operator. The detail view shown in FIG. 10 shows that when syringe plunger 12 has been depressed a desired amount, actuator 42 includes flanges 421 that engage the opposing notches 121 as driven by the biasing force provided by spring 42. Usage of this embodiment is similar to that described above and is schematically depicted in FIGS. 11-13. In particular, FIG. 11 shows syringe plunger 12 being depressed to deliver inflation fluid or gas to balloon catheter subassembly 9 (not shown in these views). When syringe plunger 12 has been advanced sufficiently as indicated in FIG. 12, flanges 421 engage notches 121 as actuator is biased outwards by spring 43, maintaining its position. As described above, this position of syringe plunger 12 corresponds to the delivery of sufficient inflation fluid or gas to inflate expandable balloon 34 so that it can be drawn into contact with bladder 3 for measurement of PUL. Plunger retracting spring 14 is now in a compressed state. Once the measurement has been recorded, the operator can depress actuator 42 as shown in FIG. 13 to overcome the biasing force of spring 43 and disengage flanges 421 from notches 121. Any suitable combination of force from retracting spring 14, manual withdrawal of syringe plunger 12 and/or elasticity of expandable balloon 34 returns the inflation fluid or gas to the inner cavity of syringe body 11 and deflates the balloon to its nominal insertion profile so that it can be removed from prostatic urethra 2.

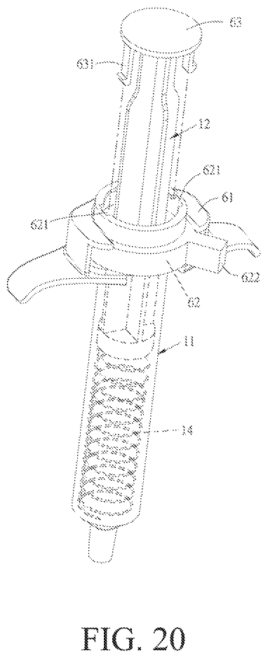

Yet another embodiment of PUL handle adapter 13 is depicted in FIG. 14. Similar to the above discussion, syringe plunger 12 has notch 121 that is engaged by syringe adapter 51 via actuator 52. Here, actuator 52 is configured as pivoting lever that is biased by leaf spring 521 to urge a projection 522 radially inwards to engage notch 121 as schematically depicted in FIGS. 15 and 16. Leaf spring 521 may be made of metal, plastic or any other material that has sufficient resilience to create the desired biasing force. Again, usage of this embodiment is similar to those described above. In particular, FIG. 17 shows syringe plunger 12 being depressed to deliver inflation fluid or gas to balloon catheter subassembly 9 (not shown in these views). When syringe plunger 12 has been advanced sufficiently as indicated in FIG. 18 having compressed retracting spring 14, actuator 52 pivots due to leaf spring 521 so that projection 522 engages notch 121 to hold syringe plunger 12 in its desired position in which expandable balloon 34 is inflated. After measurement of PUL, the operator can depress actuator 52 so that projection 522 pivots outwardly and disengages from notch 121 as shown in FIG. 19. Once more, any suitable combination of force from retracting spring 14, manual withdrawal of syringe plunger 12 and/or elasticity of expandable balloon 34 returns the inflation fluid or gas to the inner cavity of syringe body 11 and deflates the balloon to its nominal insertion profile so that it can be removed from prostatic urethra 2.

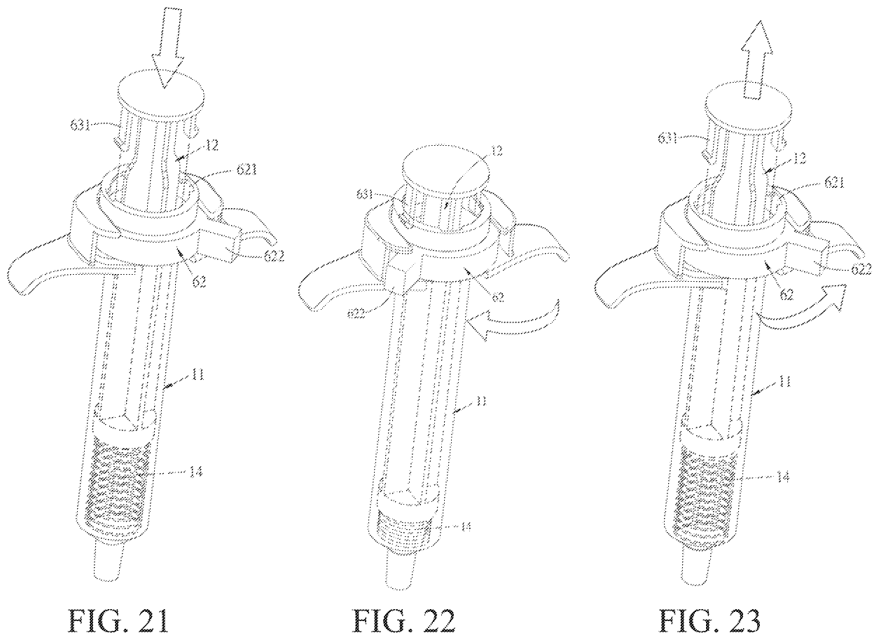

A still further embodiment of PUL handle adapter 13 is depicted in FIG. 20. As shown, syringe adapter 61 has a rotating locking ring 62 having a handle 622. A cap 63 that may be secured to the proximal end of syringe plunger 12 has one or more extension hooks 631 (two in the depicted embodiment) with which corresponding one or more notches 621 of locking ring 62 may be rotated into alignment. Cap 63 may be formed integrally with syringe plunger 12 or may be secured to a conventional syringe. Accordingly, FIG. 21 shows syringe plunger 12 being depressed to deliver inflation fluid or gas to balloon catheter subassembly 9 (not shown in these views). Notably, locking ring 62 has been rotated (using handle 622) so that notches 621 are aligned with extension hooks 631 to allow syringe plunger 12 to be depressed so that extension hooks 631 are within locking ring 62. When syringe plunger 12 has been advanced sufficiently as indicated in FIG. 22 having compressed retracting spring 14, locking ring 62 may be rotated so that notches 621 are not aligned with extension hooks 631, thereby locking syringe plunger 12 in its desired position in which expandable balloon 34 is inflated. After measurement of PUL, the operator can rotate locking ring 62 to return notches 621 into alignment with extension hooks 631 to allow retracting spring 14, either alone or in conjunction with manual withdrawal of syringe plunger 12 and/or elasticity of expandable balloon 34, to return the inflation fluid or gas to the inner cavity of syringe body 11 and deflate the balloon to its nominal insertion profile so that it can be removed from prostatic urethra 2.

The exemplary embodiments disclosed above are merely intended to illustrate the various utilities of this disclosure. It is understood that numerous modifications, variations and combinations of functional elements and features of the present disclosure are possible in light of the above teachings and, therefore, within the scope of the appended claims, the present disclosure may be practiced otherwise than as particularly disclosed and the principles of this disclosure can be extended easily with appropriate modifications to other applications.

All patents and publications are herein incorporated for reference to the same extent as if each individual publication was specifically and individually indicated to be incorporated by reference. It should be understood that although the present disclosure has been specifically disclosed by preferred embodiments and optional features, modification and variation of the concepts herein disclosed may be resorted by those skilled in the art, and that such modifications and variations are considered to be within the scope of this disclosure.

What is claimed is:

1. A measuring device for determining prostatic urethral length, comprising:
a syringe body;
a syringe plunger;
an adapter secured to the syringe body having a lock that engages the syringe plunger at a predefined position with respect to the syringe body; and
a balloon catheter subassembly that provides fluid communication between an inner cavity of the syringe body with an expandable balloon positioned at a distal end of the balloon catheter subassembly, wherein the balloon catheter subassembly further comprises markings indicating distance from the expandable balloon.

2. The measuring device of claim 1, wherein the predefined position of the syringe plunger corresponds to a desired inflation state of the expandable balloon.

3. The measuring device of claim 2, wherein the lock engages automatically when the syringe plunger is at the predefined position.

4. The measuring device of claim 3, wherein the lock comprises an actuator that is biased by a spring into engagement with at least one notch in the syringe plunger.

5. The measuring device of claim 4, wherein the actuator slides radially with respect to the syringe body and comprises at least one flange that engages the at least one notch in the syringe plunger.

6. The measuring device of claim 5, wherein the actuator comprises two flanges that are configured to engage opposing notches in the syringe plunger.

7. The measuring device of claim 4, wherein the actuator comprises a pivoting lever having a projection configured to engage the at least one notch in the syringe plunger.

8. The measuring device of claim 4, wherein manual movement of the actuator is configured to cause disengagement from the at least one notch and allow deflation of the expandable balloon.

9. The measuring device of claim 2, wherein the lock comprises an actuator and rotation of the actuator selectively engages the syringe plunger at the predefined position.

10. The measuring device of claim 9, wherein the syringe plunger has a cap with at least one extension hook and wherein the actuator has at least one notch that is rotatable into alignment with the at least one extension hook.

11. The measuring device of claim 1, wherein the adapter is a separate element that is attachable to the syringe body.

12. An adapter for a syringe having a syringe body and a syringe plunger, comprising a lock attachable to the syringe body that engages the syringe plunger at a predefined position of the syringe plunger corresponding to a desired delivery volume.

13. The adapter of claim 12, wherein the lock engages automatically when the syringe plunger is at the predefined position.

14. The adapter of claim 13, wherein the lock comprises an actuator that is biased by a spring into engagement with at least one notch formed in the syringe plunger.

15. The adapter of claim 14, wherein the actuator slides radially with respect to the syringe body and comprises at least one flange that engages the at least one notch formed in the syringe plunger.

16. The adapter of claim 14, wherein the actuator comprises a pivoting lever having a projection configured to engage the at least one notch formed in the syringe plunger.

17. The adapter of claim 12, wherein the lock comprises an actuator and rotation of the actuator selectively engages the syringe plunger at the predefined position, wherein the syringe plunger has a cap with at least one extension hook and wherein the actuator has at least one notch that is rotatable into alignment with the at least one extension hook.

18. A method for determining prostatic urethral length of a patient, comprising:
providing a balloon catheter subassembly having an expandable balloon in fluid communication with an inner cavity of a syringe body with an expandable balloon positioned at a distal end of the balloon catheter subassembly, wherein the balloon catheter subassembly further comprises markings indicating distance from the expandable balloon;
advancing the expandable balloon through the patient's prostatic urethra into the patient's bladder;
depressing a syringe plunger to inflate the expandable balloon within the patient's bladder;
locking the syringe plunger at a predefined position with respect to the syringe body with an adapter, wherein the predefined position of the syringe plunger corresponds to a desired inflation state of the expandable balloon; and
determining prostatic urethral length based at least in part on comparing the markings indicating distance from the expandable balloon to a target location of the patient.

19. The method of claim 18, wherein the syringe plunger is locked at the predefined position automatically.

20. The method of claim 18, further comprising attaching the adapter to the syringe body.

21. The method of claim 18, further comprising disengaging the lock, deflating the expandable balloon and withdrawing the balloon catheter assembly from the patient.
